(19) United States
(12) Patent Application Publication
(10) Pub. No.: US 2008/0306510 A1
(43) Pub. Date: Dec. 11, 2008

(54) FIXATION SUTURE DEVICE AND METHOD

(76) Inventor: Robert P. Stchur, Punta Gorda, FL (US)

Correspondence Address: EDWARD M. LIVINGSTON, PA, 963 TRAIL TERRACE DRIVE, NAPLES, FL, 34103, US

(21) Appl. No.: 11/758,143

(22) Filed: Jun. 5, 2007

(51) Int. Cl. A61L 17/00; A61B 17/04; A61B 17/58

(52) U.S. Cl. 606/232; 606/228

(57) ABSTRACT

A fixation suture device (12) wherein an anchor (1) has a top (6), a core (2) and a tip (5) and external threads (4) located on the anchor core (2). An anchor aperture (3) is located on the anchor (1) for acceptance of sutures (13). To use the device (12), a surgeon passes the sutures (13) through a rotator cuff tendon (14). Then, the sutures (13) are passed through the anchor aperture (3) and the anchor tip (5) is positioned against a humerus (21) via a handle (22) and cannula (23). The surgeon pushes downward on the handle (22) to provide a downward force on the anchor (1) while pulling upward on the sutures (13) in order to produce a desired tension on the rotator cuff tendon (14). The surgeon rotates the anchor (1) into the humerus (21) while maintaining tension on the sutures (13). As the anchor (1) is rotated deeper and deeper into the humerus (21), the sutures (13) secured to the rotator cuff tendon (14) wind around the anchor core (2), thereby pulling the rotator cuff tendon (14) closer and closer to the anchor (1). When the anchor top (6) is in alignment with or located below the humerus (21), the sutures (13) wrap around the sutures (13) held by the surgeon to create a wrap (24).

### BACKGROUND OF THE INVENTION

This invention relates to suture devices, more particularly, a fixation suture device that permits a user to quickly and easily fixate a suture without the need to tie conventional suture knots.

As with any medical procedure wherein a patient undergoes surgery, there is always concern relating to the amount of time the wound is exposed and the length of time spent under anesthesia. Thus, time is of the essence when operating on a patient.

Currently, arthroscopic rotator cuff operations are commonly performed for rotator cuff tears wherein a central point on the greater tuberosity is commonly found to be the symmetrical origin of the tear. The rotator cuff tear is repaired by using a plurality of sutures which are inserted through the rotator cuff tendon, usually on the greater tuberosity, wherein the sutures are ultimately knotted to maintain tension on the rotator cuff tendon, thereby permitting a person to have function of the rotator cuff tendon once again. However, the act of tying knots and overall suture management is very time-consuming. In addition, the greater the number of sutures used, the more confusion is created when ultimately tying the suture knots.

There are currently devices on the market that are designed to pass suture tension and fixate the rotator cuff tendon without having to tie suture knots. However, the current devices are complicated, unreliable and, like all current techniques, often require multiple implants. In addition, the tension applied to the rotator cuff tendon is often uneven in distribution.

Therefore, due to these inefficiencies related to the suturing and fixation of the rotator cuff tendon, surgeons spend unneeded time and energy on this procedure and, more importantly, patients are exposed to an unnecessary amount of time under anesthesia.

Thus, a need exists for a fixation suture device and method that reduces the amount of time required to perform a rotator cuff repair procedure and provides a better fixation of the rotator cuff tendon without the need to tie conventional suture knots.

The relevant prior art includes the following references:

| Patent No. | | |
|---|---|---|
| (U.S. unless stated otherwise) | Inventor | Issue/Publication Date |
| 2005/0075668 | Lizardi | Apr. 07, 2005 |
| 2006/0235413 | Denham et al. | Oct. 19, 2006 |
| 2005/0080455 | Schmieding et al. | Apr. 14, 2005 |
| 2004/0243180 | Donnelly et al. | Dec. 02, 2004 |
| 6,641,597 | Burkhart et al. | Nov. 04, 2003 |
| 2007/0005068 | Sklar | Jan. 04, 2007 |
| 2006/0161159 | Dreyfuss et al. | Jul. 20, 2006 |
| 2006/0116719 | Martinek | Jun. 01, 2006 |
| 6,117,162 | Schmieding et al. | Sep. 12, 2000 |
| 6,214,031 | Schmieding et al. | Apr. 10, 2001 |
| 6,916,333 | Schmieding et al. | Jul. 12, 2005 |
| 4,632,100 | Somers et al. | Dec. 30, 1986 |
| 5,851,219 | Goble et al. | Dec. 22, 1998 |
| 5,156,616 | Meadows et al. | Oct. 20, 1992 |
| 5,824,011 | Stone et al. | Oct. 20, 1998 |
| 7,156,864 | Linter | Jan. 02, 2007 |
| 6,027,523 | Schmieding et al. | Feb. 22, 2000 |
| 7,144,415 | Del Rio et al. | Dec. 05, 2006 |
| 7,081,126 | McDevitt et al. | Jul. 25, 2006 |
| 7,083,638 | Foerster | Aug. 01, 2006 |
| 6,780,198 | Gregoire et al. | Aug. 24, 2004 |
| 6,652,561 | Tran | Nov. 25, 2003 |
| 6,527,795 | Lizardi | Mar. 04, 2003 |

### SUMMARY OF THE INVENTION

The primary object of the present invention is to provide a fixation suture device and method that reduces the amount of time to perform a rotator cuff repair and to provide a more secure fixation of a rotator cuff tendon.

A further object of the present invention is to provide a fixation suture device and method that eliminates the need for tying suture knots.

An even further object of the present invention is to provide a fixation suture device and method that may be used on a variety of rotator cuff tear sizes.

Another object of the present invention is to provide a fixation suture device and method that reduces the amount of time the wound is exposed to the air, thereby reducing the risk of contamination and infection.

Another object of the present invention is to provide a fixation suture device and method the reduces the length of time a person is spent under anesthesia.

A further object of the present invention is to provide a fixation suture device and method that permits a user to use a large number of sutures.

An even further object of the present invention is to provide a fixation suture device and method that permits a user to tension a rotator cuff tendon and fixate at least one suture in a single movement.

Another object of the present invention is to provide a fixation suture device and method that permits adjustment of the tension on the rotator cuff tendon.

An even further object of the present invention is to provide a fixation suture device and method that requires little arthroscopic skill.

Another object of the present invention is to provide a fixation suture device and method that permits fixation of a plurality of sutures wherein each suture is fixed independently from one another.

The present invention fulfills the above and other objects by providing a fixation suture device wherein an anchor has a top, a core and a tip and at least one external thread located on the anchor core. An anchor aperture is located on the anchor, preferably on the tip of the anchor, for acceptance of at least one suture therethrough. The aperture may be of any shape or size.

To use the fixation suture device of the present invention, a surgeon first passes at least on suture end through a rotator cuff tendon at a location adjacent to a rotator cuff tear as is conventionally done when performing rotator cuff surgeries. Then, the at least one suture is passed through the anchor aperture and the tip of the anchor is positioned against a humerus, preferably via a handle and cannula. After positioning the tip of the anchor at a desired location on a humerus, the surgeon then pushes downward on the handle to provide a downward force on the anchor while pulling upward on the at least one suture in order to produce a desired tension on the rotator cuff tendon. Once the desired tension is achieved, the surgeon then rotates the anchor into the humerus while maintaining tension on the at least one suture. As the anchor is rotated deeper and deeper into the humerus, the at least one suture secured to the rotator cuff tendon wraps around the anchor core between the at least one external threads, thereby pulling the rotator cuff tendon closer and closer to the anchor. When the anchor top is in alignment with or located below the humerus, the at least one suture wraps around the at least one suture being held by the surgeon. Thus, the at least one suture is secured to the anchor and eliminates the need for the surgeon to tie a conventional suture knot. After the at least one suture is wrapped around itself and is thus secured, the surgeon cuts the excess ends of the at least one suture.

In addition, because the at least one suture is wrapped around the anchor core, the friction of the at least one suture on the anchor also aids in securing the at least one suture to the anchor.

Finally, because the anchor is installed within the humerus and the incline plane of the external threads compress the surrounding bone, the torque resistance of the anchor is increased. Thus, greater bone compression against the at least one suture is created, thereby further aiding in securement of the at least one suture to the anchor.

Alternative embodiments of the anchor include at least one anchor aperture being located on the anchor core, the anchor core being roughened and the anchor having a male or female head for acceptance of a male or female handle. In addition, notches may be used in place of the apertures.

In the alternative, a user may use the anchor in conjunction with a retaining cone having a substantially conical shape having an interior and exterior surface. A plurality of external threads are located on the exterior surface of the retaining cone while a plurality of internal threads are located on the interior surface of the retaining cone. An anchor opening is centrally located within the retaining cone for acceptance of the anchor and the internal threads encircle the anchor opening.

If using the retaining cone in conjunction with the anchor, a surgeon first passes at least one suture through the rotator cuff tendon as described above. Then, he/she installs the retaining cone into the humerus by rotating the retaining cone into the humerus. The anchor is then threaded as described above and inserted into the anchor opening. Because the internal threads of the anchor opening correspond to the external threads of the anchor, the anchor locks into the retaining cone when rotated.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a side perspective view of the fixation suture device of the present invention;

FIG. 2 is top view of the fixation suture device of the present invention;

FIG. 3 is a side perspective view of a second embodiment of the fixation suture device of the present invention;

FIG. 4 is a top view of the embodiment of FIG. 3;

FIG. 5 is a side perspective view of a third embodiment of the fixation suture device of the present invention;

FIG. 6 is a side perspective view of a fourth embodiment of the fixation suture device of the present invention;

FIG. 7 is a top view of the embodiment of FIG. 6;

FIG. 8 is a side perspective view of a fifth embodiment of the fixation suture device of the present invention;

FIG. 9 is a side perspective view of a sixth embodiment of the fixation suture device of the present invention;

FIG. 10 is a side perspective view of a seventh embodiment of the fixation suture device of the present invention;

FIG. 11 is a side perspective view of an eighth embodiment of the fixation suture device of the present invention;

FIG. 12 is a posterior plan view of a plurality of sutures being passed through a rotator cuff tendon;

FIG. 13 is posterior plan view of installing the fixation suture device of the present invention into a left humerus;

FIG. 14 is a posterior plan view of a surgeon producing a desired tension on a rotator cuff tendon;

FIG. 15A is posterior plan view of creating a wrap using the fixation suture device of the present invention;

FIG. 15B is a side perspective view of the wrap in FIG. 15A;

FIG. 16 is posterior plan view of a rotator cuff tendon secured to a humerus using the fixation suture device of the present invention;

FIG. 17 is a side view of a retaining cone used in conjunction with the fixation suture device of the present invention;

FIG. 18 is a cross-sectional view along line 18-18 of the embodiment of FIG. 17; and

FIG. 19 is a cross-sectional view of a retaining cone used in conjunction with the fixation suture device of the present invention.

### DESCRIPTION OF THE PREFERRED EMBODIMENTS

For purposes of describing the preferred embodiment, the terminology used in reference to the numbered components in the drawings is as follows:

1. anchor
2. anchor core
3. anchor aperture
4. anchor external thread
5. anchor tip
6. anchor top
7. male head
8. roughened surface
9. female head
10. anchor top thread
11. notch
12. fixation suture device,
generally
13. suture
14. rotator cuff tendon
15. rotator cuff tear
16. greater tuberosity
17. teres minor
18. infra spinatus
19. supia spinatus
20. humeral head
21. humerus
22. handle
23. cannula
24. wrap
25. retaining cone
26. retaining cone exterior surface
27. retaining cone interior surface
28. retaining cone exterior thread
29. anchor opening
30. driver opening
31. retaining cone rim
32. retaining cone interior thread

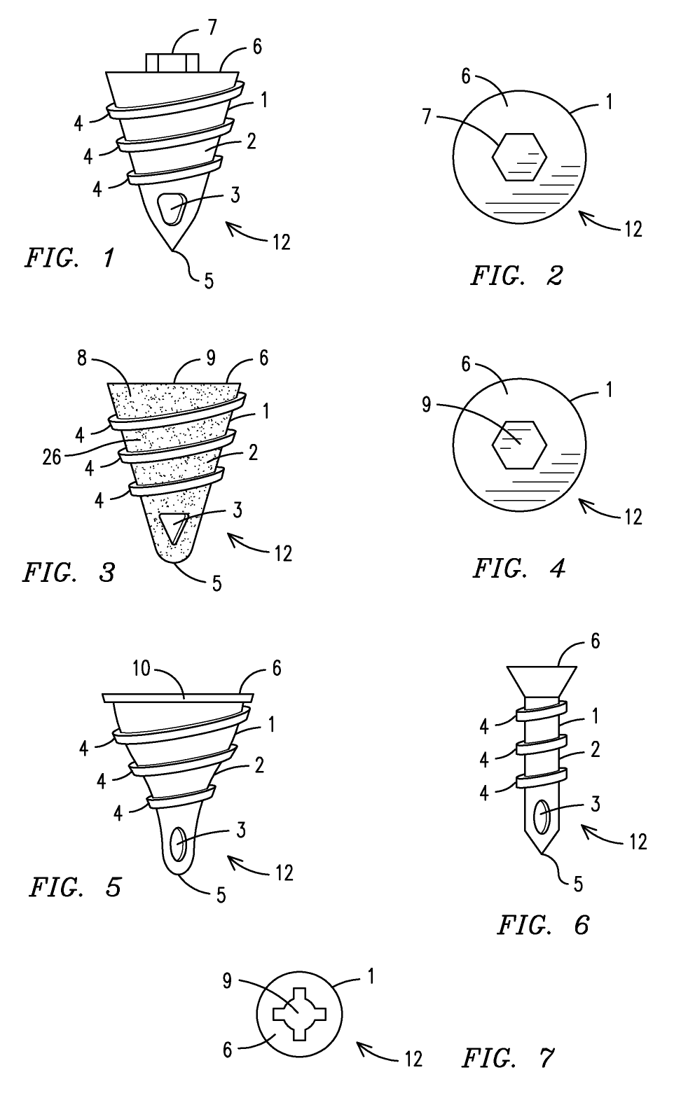

With reference to FIGS. 1 and 2, varying views of the fixation suture device of the present invention are shown. The fixation suture device 12 is an anchor 1, which is preferably tapered or conical in shape but may also be step-tapered or conical with a point, has an anchor core 2, an anchor top 6 and an anchor tip 5. The tip 5 may be pointed or sharp. At least one anchor external thread 4 is located on the anchor core 2 and winds around the anchor core 2. Preferably, three or four anchor external threads 4 around the anchor core 2; however, various numbers of anchor external threads 4 may also be used. At least one anchor aperture 3 is located on the anchor 1, preferably on the anchor tip 5. The anchor aperture 3 may be of various sizes, shapes and angles, including round, oval, triangular, etc.

A male head 7 is located on the anchor top 6 for accommodation of a handle 22 (as shown in FIG. 13) having a female head. The anchor 1 is preferably made of metal, but may also be made of other material such as bioabsorbable material. In addition, the size and pitch of the anchor external threads 4 and may be of variable size. Moreover, the taper of the anchor core 2 may be of variable size.

FIGS. 3 and 4 show varying views of a second embodiment of the fixation suture device of the present invention. Similar to the first embodiment, the fixation suture device 12 is an anchor 1 having an anchor core 2, an anchor top 6, an anchor tip 5, anchor external threads 4 and an anchor aperture 3 located on the anchor tip 5. However, in this embodiment, the anchor core 2 has a roughened surface 8 or roughened so as to increase the amount of friction between the sutures 13 and the anchor core 2. In addition, a female head 9 is located on the anchor top 6 for acceptance of a handle 22 having a male head (not shown).

With respect to FIG. 5, a side perspective view of a third embodiment of the fixation suture device of the present invention is shown. The fixation suture device 12 of the third embodiment is step-tapered and includes an anchor aperture 3 having an oval shape so as to accommodate a plurality of sutures 13 (not shown). In addition, the anchor tip 5 is rounded and an anchor top thread 10 is located at a top of the anchor core 2 adjacent to the anchor top 6 to permit a surgeon to wrap the sutures 13 around the anchor top thread 10 so the sutures 13 further wrap onto itself if advanced past the surface of the bone (not shown).

In FIGS. 6 and 7, varying views of a fourth embodiment of the fixation suture device of the present invention are shown. The fixation suture device 12 of the fourth embodiment has an elongated cylindrical anchor core 2 and a pointed anchor tip 5. In this manner, additional winds of the sutures 13 around the anchor core 2 may be achieved (not shown). Located on the anchor top 6 is a female head 9 for accommodation of a handle 22 having a male head (not shown).

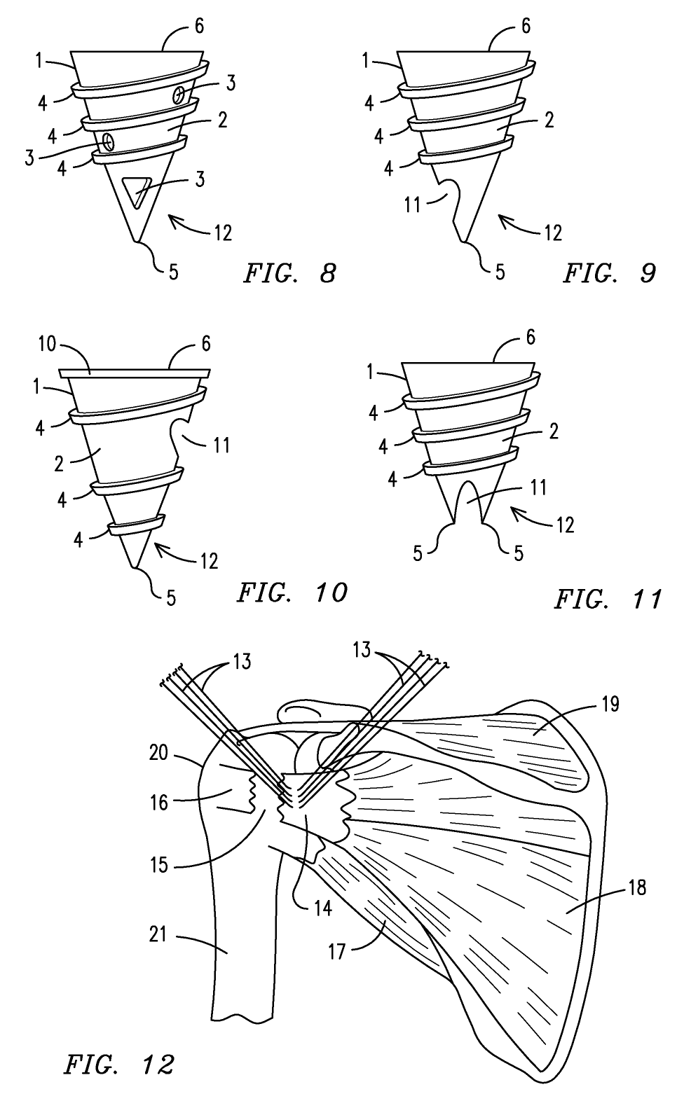

With reference to FIG. 8, a side perspective view of a fifth embodiment of the fixation suture device of the present invention is shown. Rather than having one anchor aperture 3 located on the anchor tip 5, a plurality of anchor apertures 3 are located on the anchor core 2, as well as on the anchor tip 5.

FIGS. 9-10 show perspective views a sixth, seventh and eighth embodiment of the fixation suture device of the present invention. Rather than having at least one anchor aperture 3, these embodiments of the fixation suture device 12 include at least one notch 11 located at various places on the anchor 1, including adjacent to the tip 5 (see FIG. 9), on the anchor core 2 (see FIG. 10) and on the anchor tip 5 (see FIG. 10). Similar to the aforementioned embodiments wherein at least one suture 13 is threaded through the apertures 3, at least one suture 13 is threaded through the notch 11 in these embodiments (not shown).

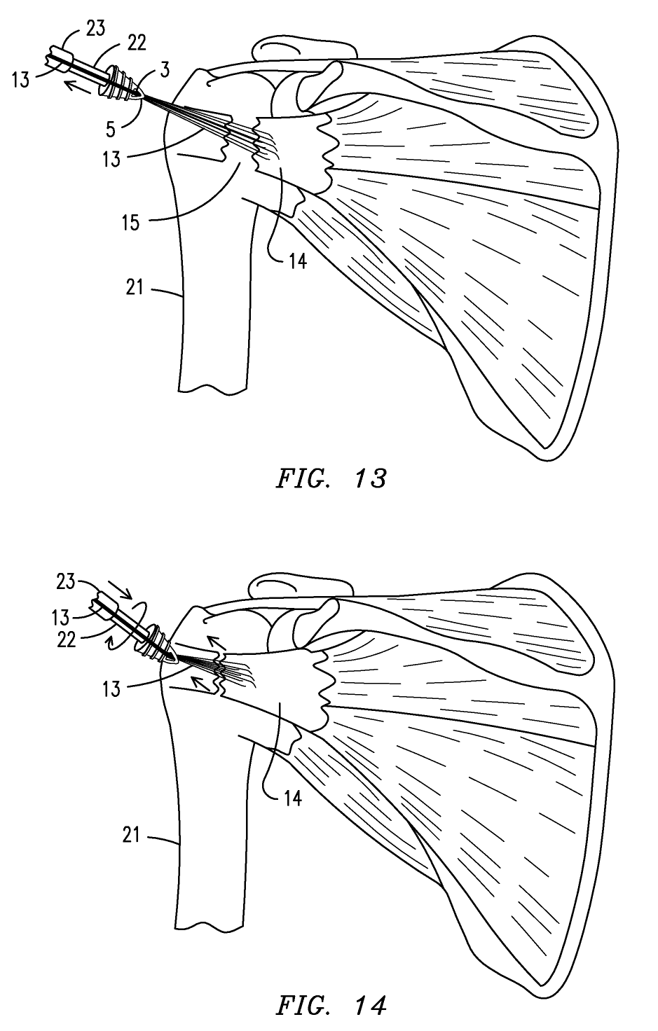

Next, FIG. 12 shows a posterior plan view of a plurality of sutures being passed through a rotator cuff tendon. The muscles located on the back of a person include the supia spinatus 19, the infra spinatus 18 and the teres minor 17 wherein a rotator cuff tendon 14 is attached to the humeral head 20 of a humerus 21 (not shown). A rotator cuff tear 15 commonly occurs in a central point on a greater tuberosity 16 of the rotator cuff tendon 14. As is conventionally performed in rotator cuff repairs, a surgeon first passes at least one suture 13 through the rotator cuff tendon 14 at a desired location using a suture passing instrument of his/her choice (not shown).

With reference to FIG. 13, a posterior plan view of installing the fixation suture device of the present invention into a left humerus is shown. Once the sutures 13 are passed through the rotator cuff tendon 14, the surgeon then secures the anchor 1 to the handle 22, which is preferably located within a cannula 23. Then, he/she passes the sutures 13 through the anchor aperture 3 and pulls the sutures 13 in order to maintain tension on the sutures 13.

After positioning the anchor tip 5 at a desired location on a humerus 21, the surgeon then pushes downward on the handle 22 to provide a downward force on the anchor 1 while pulling upward on the sutures 13 in order to maintain a desired tension on the rotator cuff tendon 14 as shown in FIG. 14. Once the desired tension is achieved, the surgeon then rotates the anchor 1 into the humerus 21 while maintaining tension on the sutures 13. In order to initially rotate the anchor 1 into the humerus 21, a surgeon may need to gently tap the handle 22 with a mallet; however, the use of a mallet is not required.

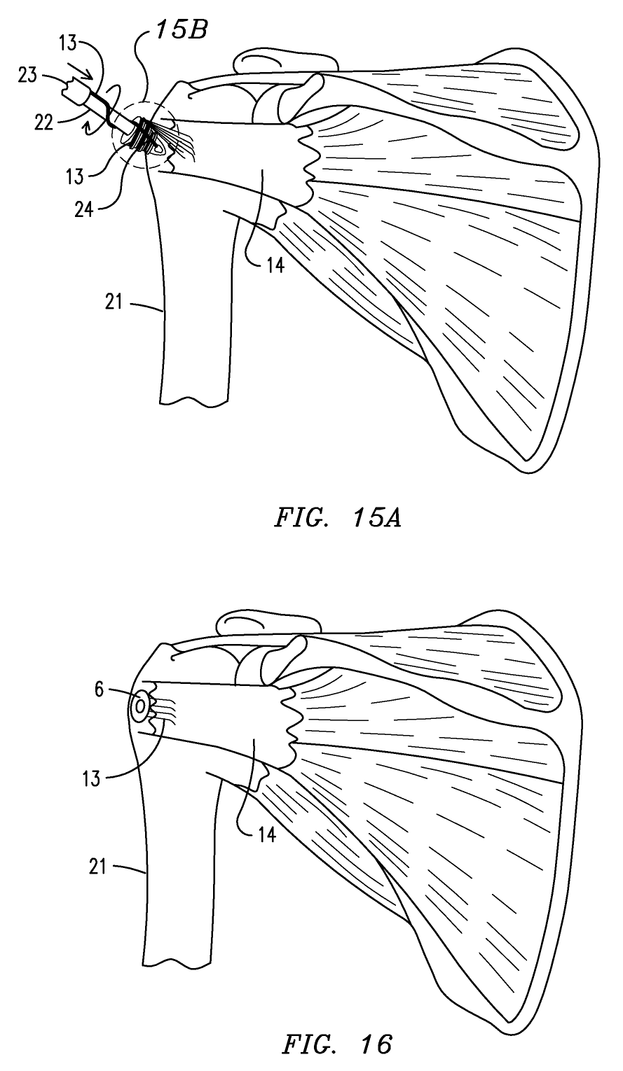

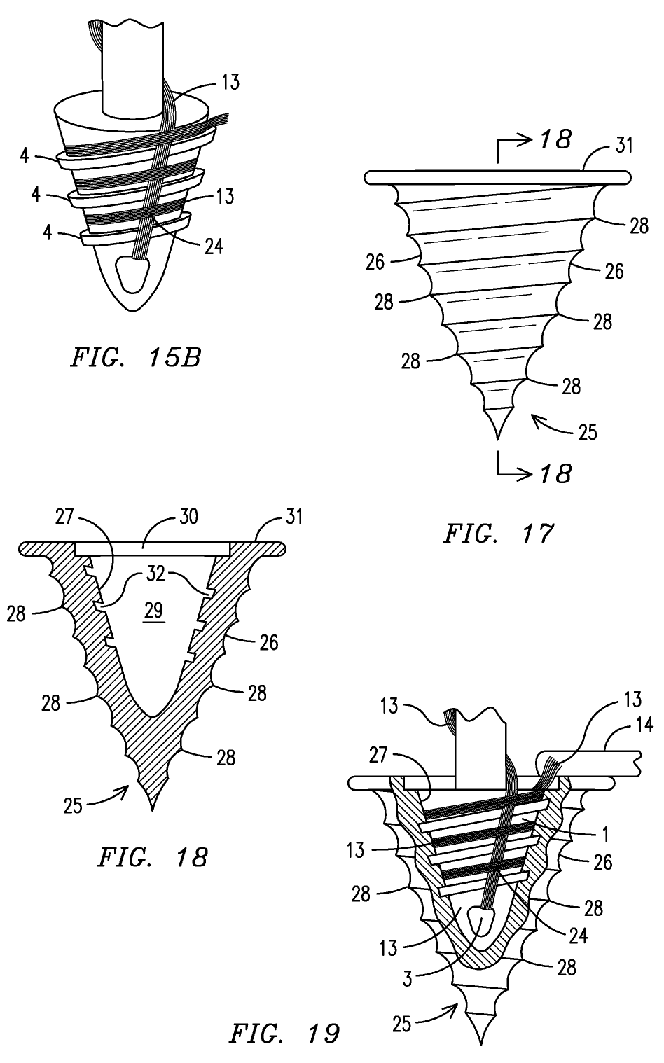

As the anchor 1 is rotated deeper and deeper into the humerus 21 as shown in FIG. 15A, the sutures 13 secured to the rotator cuff tendon 14 wind around the anchor core 2 between the external threads 4, thereby pulling the rotator cuff tendon 14 closer and closer to the anchor 1. The surgeon then rotates the anchor 1 clockwise or counterclockwise to increase or decrease the amount of tension placed on the rotator cuff tendon 14. When the anchor top 6 is in alignment with or located below the humerus 21, the sutures 13 wrap around the sutures 13 being held by the surgeon, thereby creating a wrap 24. Thus, the sutures 13 are secured to the anchor 1 via the wrap 24 and eliminate the need for the surgeon to tie a conventional suture knot. An enlarged view of the wrap 24 formed is shown in FIG. 15B.

After formation of the wrap 24, the surgeon cuts the excess ends of the sutures 13 to result in a sutured and fixated rotator cuff tendon 14 as shown in FIG. 16. Because the sutures 13 are wound around the anchor core 2, when the anchor 1 is located within the humerus 21, the sutures 13 are compressed by the humerus 21, thereby assisting in fixation of the sutures 13.

With respect to FIG. 17, a side view of a retaining cone used in conjunction with the fixation suture device of the present invention is shown. A retaining cone 25 includes a retaining cone rim 31 and has a retaining cone exterior surface 26. At least one retaining cone exterior thread 28 is located on the retaining cone exterior surface 26 and winds around the retaining cone 25. The retaining cone 25 is preferably conical in shape.

In FIG. 18, a cross-sectional view along line 18-18 of the embodiment of FIG. 17 is shown. The retaining cone 25 has a retaining cone exterior surface 26 and a retaining cone interior surface 27 wherein at least one retaining cone internal thread 32 is located on the retaining cone interior surface 27. Centrally-located within the retaining cone 25 is an anchor opening 29 having a predetermined size and shape. The internal threads 32 encircle the anchor opening 29 for accommodation of an anchor 1 as shown in FIG. 19. A driver opening 30 is located at a top of the retaining cone 25 for acceptance of a driver head when installing the retaining cone 25 within a humerus 21 (not shown).

Finally, FIG. 19 shows a cross-sectional view of a retaining cone used in conjunction with the fixation suture device of the present invention. When the anchor 1 is located within the retaining cone 25, the sutures 13, which are first threaded through the aperture 3, are also located within the retaining cone 25. The anchor 1 is rotated within the anchor opening 29 such that the anchor external threads 4 interlock with the retaining cone internal threads 32 in order to give an interference fit between the anchor 1 and the retaining cone 25 as well as creating a wrap 24 of the sutures 13 around the anchor 1.

The use of the present invention will allow a surgeon to reduce the amount of time to perform a rotator cuff repair procedure and to provide a better fixation of the rotator cuff tendon.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A fixation suture device comprising:
an anchor having a top, core and tip;
at least one anchor external thread located on said anchor core;
at least one anchor aperture located on said anchor; and
at least one suture for threading into said at least one anchor aperture wherein said at least one suture winds around said at least one anchor external thread when said anchor is rotated.

2. The fixation suture device of claim 1 wherein:
said at least one anchor aperture is located on said anchor tip.

3. The fixation suture device of claim 1 wherein:
said at least one anchor aperture is located on said anchor core.

4. The fixation suture device of claim 2 further comprising:
at least one anchor aperture located on said anchor core.

5. The fixation suture device of claim 1 wherein:
said at least one anchor aperture is round.

6. The fixation suture device of claim 1 wherein:
said at least one anchor aperture is oval.

7. The fixation suture device of claim 1 wherein:
said at least one anchor aperture is triangular.

8. The fixation suture device of claim 1 wherein:
said anchor core is roughened.

9. The fixation suture device of claim 1 wherein:
said anchor tip is pointed.

10. The fixation suture device of claim 1 wherein:
said anchor tip is rounded.

11. The fixation suture device of claim 1 further comprising:
a female head located on said anchor top.

12. The fixation suture device of claim 1 further comprising:
a male head located on said anchor top.

13. A fixation suture device comprising:
an anchor having a top, core and tip;
at least one anchor external thread located on said anchor core;
at least one notch located on said anchor; and
at least one suture for threading into said at least one notch wherein said at least one suture winds around said at least one anchor external thread when said anchor is rotated.

14. The fixation suture device of claim 13 wherein:
said at least one notch is located on said anchor tip.

15. The fixation suture device of claim 13 wherein:
said at least one notch is located on said anchor core.

16. The fixation suture device of claim 14 further comprising:
at least one notch located on said anchor core.

17. The fixation suture device of claim 13 wherein:
said anchor core is roughened.

18. The fixation suture device of claim 1 further comprising:
a substantially conically-shaped retaining cone having an exterior and interior surface;
a rim of a predetermined shape located on said retaining cone;
at least one external thread located on said retaining cone exterior surface;
an anchor opening centrally located within said retaining cone;
at least one rim opening located on said rim wherein said rim opening provides access to said anchor opening; and
at least one internal thread located on said retaining cone interior surface and encircling said anchor opening wherein said at least one external thread on said anchor body corresponds with said at least one internal thread located on said retaining cone interior surface to lock said anchor into said retaining cone when said anchor is rotated within said retaining cone.

19. The fixation suture device of claim 13 further comprising:
a substantially conically-shaped retaining cone having an exterior and interior surface;
a rim of a predetermined shape located on said retaining cone;
at least one external thread located on said retaining cone exterior surface;
an anchor opening centrally located within said retaining cone;
at least one rim opening located on said rim wherein said rim opening provides access to said anchor opening; and
at least one internal thread located on said retaining cone interior surface and encircling said anchor opening wherein said at least one external thread on said anchor body corresponds with said at least one internal thread located on said retaining cone interior surface to lock said anchor into said retaining cone when said anchor is rotated within said retaining cone.

20. A method for using a fixation suture device comprising an anchor having a top, core and tip; at least one anchor external thread located on said anchor core; at least one anchor aperture located on said anchor; and at least one suture for threading into said at least one anchor aperture wherein said at least one suture winds around said at least one anchor external thread when said anchor is rotated, said method comprising steps of:
a. passing said at least one suture through a rotator cuff tendon;
b. passing said at least one suture through said at least one aperture;
c. pulling upward on said at least one suture in order to produce a desired tension on said rotator cuff tendon; and
d. rotating said anchor into a humerus such that said at least one suture forms a wrap.

21. The method of claim 20 further comprising a step after step d of:
e. cutting said at least one suture.
